(19) United States
(12) Patent Application Publication
(10) Pub. No.: US 2026/0224861 A1
(43) Pub. Date: Aug. 6, 2026

(54) APPARATUS FOR AUTOMATIC INFLATION OF A VASCULAR BALLOON CATHETER, AND A SYSTEM FOR PERFORMING AN ANGIOPLASTY

(71) Applicant: B. Braun Melsungen AG, Melsungen (DE)

(72) Inventors: Felix Fulda, Berlin (DE); Andreas Stolle, Berlin (DE)

(21) Appl. No.: 19/153,076

(22) PCT Filed: Feb. 1, 2024
(86) PCT No.: PCT/EP2024/052557

§ 371 Date: Aug. 1, 2025

(30) Foreign Application Priority Data
Feb. 2, 2023 (DE) 10 2023 102 573.2

(51) Int. Cl. A61M 25/10

(52) U.S. Cl. CPC A61M 25/10182; A61M 25/10188; A61M 25/104; A61M 2025/1077; A61M 2205/3327; A61M 2205/3331; A61M 2205/505; A61M 2205/8275

(57) ABSTRACT

A device for automatically inflating a vascular balloon catheter with a fluid includes a fluid delivery device, a pressure sensor device, and a control device. A catheter interface is connectable to the balloon catheter in a demountable manner so that the fluid can flow from the device into the balloon catheter via the catheter interface. The fluid delivery device is configured to eject the fluid from the device at the catheter interface via a motor. The pressure sensor device is configured to measure a pressure of the fluid ejected from the device. The control device is configured to control the amount of fluid ejected from the device via the fluid delivery device as a function of the pressure measured by the pressure sensor device by driving the motor of the fluid delivery device.

### CROSS-REFERENCE TO RELATED APPLICATIONS

This application is the United States national stage entry of International Application No. PCT/EP 2024/052557, filed on Feb. 1, 2024, and claims priority to German Application No. 10 2023 102 573.2, filed on Feb. 2, 2023. The contents of International Application No. PCT/EP 2024/052557 and German Application No. 10 2023 102 573.2 are incorporated by reference herein in their entireties.

### FIELD

The present disclosure relates to a device for automatically inflating a vascular balloon catheter, in particular a vascular angioplasty catheter and a percutaneous transluminal angioplasty catheter, respectively (for PTA: percutaneous transluminal angioplasty or PTCA: percutaneous transluminal coronary angioplasty) by means of a fluid. The device has a catheter interface, a fluid delivery device, a pressure sensor device, and a control device. The catheter interface is designed so that it can be connected to the balloon catheter in a demountable manner, in particular without tools, thus allowing the fluid to flow from the device via the catheter interface into the balloon catheter. The fluid delivery device is designed so as to be able to eject the fluid from the device at the catheter interface by means of a motor. The pressure sensor device is adapted to measure the pressure of the fluid ejected from the device. The control device is adapted to be able to control the amount of fluid ejected from the device by the fluid delivery device as a function of the pressure measured by the pressure sensor device by controlling the motor of the fluid delivery device. The present disclosure further relates to a system for performing angioplasty, comprising a device according to the disclosure and a balloon catheter.

### BACKGROUND

A typical device is disclosed, for example, in EP 0 490 979 B1. The conventional device has a catheter interface, a fluid delivery device, a pressure sensor device, and a control device.

The fluid delivery device is adapted to deliver a fluid via the catheter interface into a balloon catheter. In order to control the inflation of the balloon catheter resp. a balloon of the balloon catheter, a pressure in the balloon catheter resp. in the balloon can be measured by way of the pressure sensor device. The control device is adapted to control the fluid delivery device as a function of the pressure measured by the pressure sensor device. In order to be able to deliver the fluid from the balloon catheter, the conventional device has a piston syringe that only can be operated manually.

Inflating the balloon of the balloon catheter during angioplasty causes an interruption in the blood supply. The problem with the conventional device is that the interruption of blood flow can be excessively long and, if excess pressure occurs in the balloon catheter, the excess fluid causing the excess pressure can only be removed from the balloon catheter with a certain time delay.

CN 108096676 A discloses a tracheal catheter, i.e., a catheter for the trachea. This balloon operates with overpressures of approximately 25 to 30 mbar.

DE 10 2005 060 197 A1 discloses a completely implanted device. For this reason, this device cannot evacuate a balloon catheter, as evacuated gas would be pumped back into the balloon when it is inflated. Furthermore, it is a flushing catheter with a latex balloon that is operated with only minimal overpressures of a few millibars.

DE 10 2014 003 306 A1 discloses a balloon catheter for trans-anal irrigation. Also for such a balloon catheter, evacuation is not provided.

US 2007 0 197 963 A1 discloses a device for applying an already evacuated balloon catheter. The device is not intended and not capable, either, of evacuating a balloon catheter, as it primarily uses a peristaltic pump. However, such a pump cannot generate a vacuum because the peristaltic tube would not “reposition itself” when negative pressure is built up. However, if the tube is stiffened to achieve this property the tube can no longer be compressed and therefore it can no longer be pumped. A peristaltic pump can only generate minimal negative pressures.

According to US 2007 0 197 963 A1, it is only the balloon of a balloon catheter that is emptied and not the catheter of the balloon catheter.

U.S. Pat. No. 5,599,301 A1 discloses an automatic inflation device for a catheter system for use in a body lumen of a patient. The automatic inflation device comprises a catheter with a proximal and a distal end and an inflatable element near the distal end, as well as an inflation lumen within the catheter to establish a fluid connection between the proximal end and the inflatable element. The automatic inflation device further comprises a pump suitable for pressurising fluid in the inflation lumen and the inflatable element, the pump having an associated movement speed and movement direction. In addition, the automatic inflation device comprises a pressure sensor suitable for measuring the fluid pressure in the inflatable element and a controller adapted to be programmed with a selected rate of fluid pressure change. The controller receives the measured pressure detected by the pressure sensor and determines the actual rate of pressure change therefrom. It compares the actual rate of pressure change with the programmed selected rate of change and determines the difference, and controls the speed of the pump to reduce the difference, thereby inflating the inflatable element with fluid pressurised at the selected rate of pressure change.

### SUMMARY

The purpose of the present disclosure therefore is to provide a device for automatically inflating a balloon catheter, which makes it possible to minimize the duration of an interruption in blood supply and/or to reduce the amount of fluid in the balloon catheter in a timely manner.

A device according to the disclosure is suitable for automatically inflating a balloon catheter with a fluid. The fluid may be a liquid, in particular a saline solution and/or a contrast agent. Examples of contrast agents include iopromide, iodixanol, ioxaglate, iohexol, iopamidol, iomeprol, iomeron, gadodiamide, or gadolinium. For dilution, the contrast agent is preferably diluted with saline solution, e.g., 0.9% of NaCl. Gadodiamide or gadolinium may also be used undiluted. A mixing ratio of contrast agent to diluent of 1:1 to 1:3, preferably approximately 1:2, is suitable.

The device has a catheter interface, a fluid delivery device, a pressure sensor device, and a control device.

The catheter interface is designed such that it can be connected to the balloon catheter in a demountable manner, in particular without tools, so that the fluid can flow from the device via the catheter interface into the balloon catheter. The catheter interface can be designed in particular as a Luer connection.

The fluid delivery device is designed so as to be able to eject the fluid from the device at the catheter interface by means of a motor.

The fluid delivery device can be designed in particular as a piston syringe, and a piston of the piston syringe can be connected to a motor such that the piston can be moved by way of the motor. The motor can be configured as a linear motor that transmits a linear movement directly to the piston. Alternatively, the motor may also be designed as a rotary motor. In this case, a transmission can be provided by means of which a rotary movement of the motor can be converted into a linear movement, resulting in a displacement of the piston.

Optionally, another way of transmission can be used for the linear movement of the piston, such as a toggle lever, scissor joint, or a lever construction for converting the rotary movement of the motor into a linear movement of the piston.

The motor can be, in particular, an electric, mechatronic, pneumatic, or hydraulic motor.

The pressure sensor device is adapted to measure the pressure of the fluid ejected from the device. In particular, the pressure sensor device can be adapted to measure the pressure of the fluid ejected from the device at the catheter interface and/or at the piston of the piston syringe and/or at another suitable location. Preferably, a pressure sensor (as part of the pressure sensor device) can be provided at the catheter interface, in particular on an outer side of the device, which pressure sensor, in a state in which a balloon catheter is connected to the device resp. to the catheter interface of the device, is in fluid communication with the balloon catheter or the balloon of the balloon catheter. The pressure sensor device or the pressure sensor can be connected to the control device by a cable or in a wireless manner.

The control device is adapted to control a quantity of the fluid ejected from the device by way of the fluid delivery device directly (by monitoring the quantity) or indirectly (by monitoring a delivery rate of the fluid delivered by the fluid delivery device) as a function of the pressure measured by the pressure sensor device, by driving the motor of the fluid delivery device. In particular, the control device can be designed to reach a predetermined target pressure in the balloon catheter or in the balloon of the balloon catheter by conveying a corresponding amount of fluid from the device into the balloon catheter.

According to the disclosure, the fluid delivery device is designed to be able to suck in the fluid into the device at the catheter interface by means of the motor.

In other words, the fluid delivery device is designed in a state, in which a balloon catheter is connected to the device resp. to the catheter interface of the device, to optionally deliver fluid from the device into the balloon catheter resp. the balloon of the balloon catheter, or to convey fluid from the balloon catheter resp. the balloon of the balloon catheter into the device.

The device can be adapted to form a closed system with the balloon catheter when connected to the balloon catheter, i.e., fluid is only transferred between the device and the balloon catheter as required. Alternatively, the device can also be adapted to form an open system with the balloon catheter when connected to the balloon catheter. In particular, in the case of an open system, the device can have a fluid inlet for connecting the device to a fluid source and a fluid outlet for connecting the device to a fluid discharge.

Due to the fact that the device according to the disclosure is designed to be able to optionally eject or suck in fluid by means of the delivery device, it is advantageously possible to quickly change the volume of a balloon of a balloon catheter during angioplasty.

The device disclosed in the present disclosure is capable of initially evacuating a balloon catheter, i.e., applying a strong negative pressure to the applied balloon catheter in order to remove any residual air remaining in the balloon catheter after flushing the balloon catheter and, above all, to prevent it from re-entering the balloon catheter. The air interferes with inflation and is particularly dangerous if the balloon catheter bursts. This is because, unlike the inflation fluid, residual air in the balloon catheter would expand explosively at pressures of 10 bar and above and, in the worst case, injure or even rupture the vessel. In addition, air in the blood vessels may lead to thrombosis.

The delivery device can be designed to eject fluid at a rate of 0.33 ml/s or more (1 ml/s) and to suck in fluid at a rate of 10 to 15 ml/s.

According to one aspect of the disclosure, the control device can be designed to cause the fluid to be sucked in by way of the fluid delivery device when a predetermined threshold value of the pressure measured by the pressure sensor device is exceeded. The predetermined threshold value may correspond to a target pressure that is to be provided during proper performance of an angioplasty. Alternatively, the predetermined threshold value may also be greater than this target pressure, so that, for performing an angioplasty with the target pressure and the predetermined threshold value, a permissible pressure range or a target pressure range is defined.

If a predetermined threshold value is specified, which, when exceeded, automatically causes suction of the fluid, safety during angioplasty can be increased.

According to one aspect of the disclosure, the control device can be designed to prevent the fluid from being sucked in by the fluid delivery device after the predetermined threshold value has been exceeded and a subsequent drop in the pressure measured by the pressure sensor device when the predetermined threshold value is reached again.

If the device is designed such that suction of the fluid is only initiated when a predetermined threshold value is exceeded, it is advantageously possible to reduce or completely eliminate excessive stress on body tissue of a body lumen during movement of a balloon catheter connected to the device without excessively reducing the force required for dilating the body lumen.

According to one aspect of the disclosure, the fluid may be a liquid and the device may comprise a venting device by means of which a gas mixed with the liquid can be discharged from the device. The venting device may be provided in particular at the catheter interface. Alternatively, the venting device may also be formed on the fluid delivery device.

If liquid is used to inflate the balloon catheter, force transmission from the motor via the fluid to a tissue to be dilated can be improved. If a venting device is provided, this improved force transmission can be ensured in an advantageous manner.

According to one aspect of the disclosure, the control unit can be designed to be able to control or to control the fluid delivery device such that the fluid is delivered at different predetermined delivery rates. In particular, different target delivery rate profiles can be stored in the control device. Preferably, a delivery rate during suction of the fluid can be greater than a delivery rate during ejection of the fluid.

If different predetermined delivery rates are provided, it is possible to adequately adjust the response times of the device to different requirements during angioplasty.

According to one aspect of the disclosure, the control unit can be designed to regulate the fluid delivery device based on the pressure measured by the pressure sensor device. In particular, the control unit can be designed so that the pressure measured by the pressure sensor device can reach at least two different predetermined pressure levels. Preferably, different pressure levels and/or predetermined sequences of pressure levels can be stored in the control device.

According to one aspect of the disclosure, the device may comprise an energy storage device (for storing electrical or mechanical energy) so that, in the event of a power failure, not only the excess pressure can be reduced, but also a vacuum can be created again, so that the balloon catheter can be deflated and/or explanted even in this case.

According to one aspect of the disclosure, the motor of the fluid delivery device may comprise tensioning means, which are designed to be tensioned when the fluid is ejected and to be relaxed when the fluid is sucked in. The tensioning means may in particular be a spring, preferably a compression spring. In particular, the fluid delivery device can be designed as a piston syringe, and a compression spring may be provided or attached to a piston of the piston syringe in such a way that the compression spring is tensioned when the piston is moved to eject the fluid from the piston syringe (by the motor), and is relaxed when the piston is moved to suck in the fluid in the piston syringe.

If tensioning means are provided in the motor, operational safety of the device can be improved. The tensioning means are capable of causing the fluid to be sucked into the device in the event of an interruption in the power supply to the motor.

According to one aspect of the disclosure, the control device may have a data interface that is designed to transmit data to and/or from a smartphone, tablet, or other device, in particular a medical device. In particular, the data interface can be adapted to enable a wired or wireless connection to a smartphone, tablet, or other medical device. Preferably, the data interface can be designed to enable a connection using a transmission standard such as USB, Wi-Fi, NFC, Bluetooth (BT), or Bluetooth Low Energy (BLE).

Alternatively, the data interface can be configured as a pressure connection and the control device can be designed to control the fluid delivery device depending on the pressure measured at the pressure connection.

If a data interface is provided operation and/or monitoring of the device according to the disclosure can be implemented in a cost-effective manner.

According to one aspect of the disclosure, the device may comprise an operating unit connected to the control device resp. the data interface, which is adapted to receive control commands from a person and transmit them to the control device. The operating unit can be connected to the control device or data interface wired or wirelessly.

If the device is equipped with an operating unit, operability of the device independent of peripheral devices can be safeguarded in an advantageous manner.

According to one aspect of the disclosure, the operating unit may be designed as a manual inflation syringe. In particular, the operating unit may have a pistol grip or a toggle-shaped handle. Preferably, the operating unit may have a slide switch or a toggle switch by means of which the predetermined threshold value for the pressure of the ejected fluid and/or the target pressure can be specified.

If the operating unit is designed in the form of a manual inflation syringe, it is easier to become accustomed to the device according to the disclosure.

According to one aspect of the present disclosure, the operating unit may be an inflation syringe or a piston syringe by means of which a control fluid can be delivered to a control fluid interface of the inflation syringe or piston syringe by moving a piston, the device may have a pressure connection designed to be connected to the control fluid interface of the inflation syringe or piston syringe, the pressure sensor device may be designed, in particular at the pressure connection, to measure a pressure of the control fluid, and the control device may be adapted to control the fluid delivery device based on the pressure of the control fluid measured, in particular at the pressure connection. The control fluid interface can be configured in particular as a Luer connection.

If the data interface is designed as a pressure connection that is compatible with a control fluid interface of an inflation syringe or piston syringe, the operating unit can be implemented in a cost-effective manner.

According to one aspect of the disclosure, the operating unit may have a display that is adapted to display condition data of the device. In particular, the operating unit can be designed as a touch-sensitive screen.

Moreover, the disclosure relates to a system for performing angioplasty, in particular percutaneous transluminal angioplasty (PTA) or percutaneous transluminal coronary angioplasty (PTCA) with a balloon catheter and a device according to the disclosure.

According to one aspect of the disclosure, the device may be designed to create a negative pressure or vacuum in the balloon catheter (i.e., in the balloon and/or in the catheter of the balloon catheter) in order to evacuate the gas or liquid in the catheter from the catheter.

### BRIEF DESCRIPTION OF THE DRAWINGS

The present disclosure will be described in more detail below with reference to preferred embodiments and with reference to the accompanying drawings.

FIG. 1 shows a schematic view of a system according to a first embodiment of the present disclosure.

FIG. 2 shows a schematic view of a fluid delivery device according to the present disclosure.

FIG. 3 shows a schematic view of a system according to a second embodiment of the present disclosure.

FIG. 4 shows a schematic view of a system according to a third embodiment of the present disclosure.

FIG. 5 shows a schematic view of an electrical operating unit according to a first embodiment of the present disclosure.

FIG. 6 shows a schematic view of an electrical operating unit according to a second embodiment of the present disclosure.

FIG. 7 shows a schematic view of an electrical operating unit according to a third embodiment of the present disclosure.

FIG. 8 shows a schematic view of a system according to a fourth embodiment of the present disclosure.

FIG. 9 shows a schematic view of a fluid mechanical operating unit according to a first embodiment of the present disclosure with a quick-release valve in a deactivated position.

FIG. 10 shows a schematic view of the fluid-mechanical operating unit according to the first embodiment with the quick-release valve in an activated position.

FIG. 11 shows a schematic view of a fluid-mechanical operating unit according to a second embodiment of the present disclosure.

FIG. 12 shows a schematic view of a fluid-mechanical operating unit according to a third embodiment of the present disclosure with a quick-release valve in a deactivated position.

FIG. 13 shows a schematic view of the fluid-mechanical operating unit according to the third embodiment of the present disclosure with the quick-release valve in an activated position.

### DETAILED DESCRIPTION

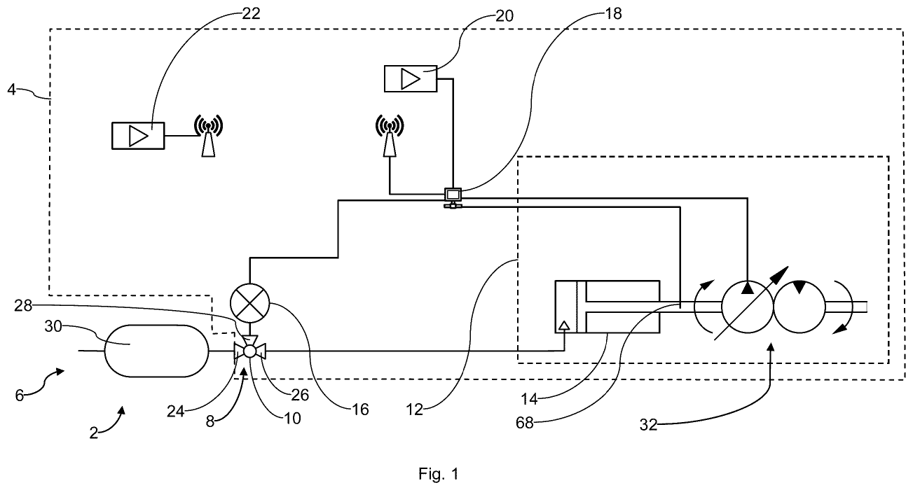

FIG. 1 shows a schematic view of a system 2 according to a first embodiment of the disclosure with a device 4 and a balloon catheter 6.

The device 4 has a catheter interface 8 in the form of a three-way valve 10, a fluid delivery device 12 in the form of a motorized piston syringe 14, a pressure sensor device 16, a control device 18, and two operating units 20 and 22. The catheter interface 8 does not have to be designed as a three-way valve 10, but can also be formed by a simple valve, for example.

The catheter interface 8 or the three-way valve 10 is designed such that the balloon catheter 6 can be connected to the catheter interface 8 and the three-way valve 10, respectively, without tools and can be disconnected from the catheter interface 8 resp. the three-way valve 10 without tools. The three-way valve 10 has three connection points 24, 26, and 28. The connection point 24 is formed in particular as a Luer connection point that is compatible with a Luer connection point on the balloon catheter 6.

The connection point 26 is fluid-mechanically connected to the piston syringe 14 of the fluid delivery device 12.

The connection point 28 is fluid-mechanically connected to the pressure sensor unit 16. If the catheter interface 8 is configured as a simple valve, no connection point 28 is provided. In this case, the pressure is measured before or after the catheter interface 8.

The control device 18 is connected to the fluid delivery device 12 and the pressure sensor device 16 at least by way of signals. Preferably, the fluid delivery device 12 and the pressure sensor device 16 also are supplied with electrical power via the control device 18.

The operating units 20 and 22 are designed that a person can give control commands or specifications to the control device 18 and receive status information about the device 4. For this purpose, the operating unit 20 is connected to the control device 18 via a cable, at least by signals. The operating unit 20 also can be connected to the control device 18 such that the operating unit 20 is or can be supplied with electrical power via the control device 18. The operating unit 22 is connected or connectable to the control device 18 via radio.

In the state shown in FIG. 1, a balloon 30 of the balloon catheter 6 is filled with fluid.

The pressure prevailing in the balloon 30 can be determined by means of the pressure sensor device 16, which is in fluid connection with the balloon 30 via the connection points 24 and 28 of the catheter interface 8 and the three-way valve 10, respectively. In a system according to the first embodiment, the pressure sensor device 16 is designed as a single pressure sensor.

The pressure determined by the pressure sensor device 16 can be output by the control device 18 and the operating unit 20 and/or 22 and a corresponding output device, respectively, for example in the form of a display.

By way of the operating unit 20 or 22 a threshold value and/or a target value can be specified for the pressure determined by way of the pressure sensor unit 16.

The control device 18 is designed to control the delivery device 12 in accordance with the specified threshold value and/or target value.

If the pressure in the balloon 30 determined by the pressure sensor device 16 is less than a target value, the control device 18 drives the fluid delivery device 12 such a that fluid flows from the fluid delivery device 12 via the connection points 26 and 24 of the catheter interface 8 to the balloon catheter 6 and the balloon 30, respectively.

In case the pressure in the balloon 30 determined by the pressure sensor device 16 is greater than or equal to the target value and less than or equal to the threshold value the control device 18 drives the fluid delivery device 12 such that no fluid flows from or to the balloon catheter 6 and the balloon 30, respectively.

If the pressure in the balloon 30 determined by the pressure sensor device 16 is larger than the threshold value, the control device 18 drives the fluid delivery device 12 such that fluid flows from the balloon 30 and balloon catheter 6, respectively, via the connection points 24 and 26 of catheter interface 8 to or into the fluid delivery device 12.

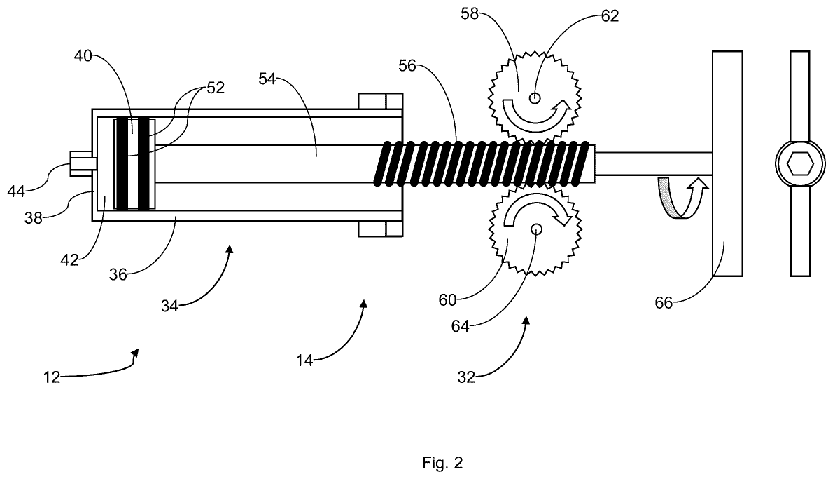

FIG. 2 shows a schematic view of the fluid delivery device 12. The fluid delivery device 12 comprises the piston syringe 14 and a motor unit or motor 32.

The piston syringe 14 has a hollow cylinder 34 with a surrounding wall 36. The hollow cylinder 34 is open on one side and has a front wall 38 on the side opposite the open side. A piston 40 is introduced into the hollow cylinder 34 on the open side of the hollow cylinder 34.

The piston 40 is displaceable in the hollow cylinder 34 and, together with the surrounding wall 36 and the front wall 38, encloses a fluid chamber 42. The size of the fluid chamber 42 is variable due to the displaceability of the piston 40 relative to the hollow cylinder 34.

A fluid outlet 44 is formed on the front wall 38. The fluid outlet 44 is connected in a fluid-mechanical manner to the connection point 26 of the catheter interface 8 and the three-way valve 10, respectively.

When the piston 40 is moved toward the front wall 38, fluid located in the fluid chamber 42 is conveyed from the fluid outlet 44 to the connection point 26 resp. the catheter interface. Moving the piston 40 toward the front wall 38 thus causes the fluid to be ejected at the catheter interface 8 resp. its connection point 24.

If the piston 40 is moved away from the front wall 38, a negative pressure is generated at the fluid outlet 44, which effects fluid to be sucked in from the connection point 26 and the catheter interface 8, respectively, and thus causes fluid to be sucked in at the catheter interface 8 resp. its connection point 24. If the balloon catheter 6 is connected to the catheter interface, moving the piston away from the front wall causes fluid to be sucked in from the balloon catheter 6 resp. the balloon 30 to the connection point 24.

Seals 52 are provided on a circumferential surface of the piston 40 between the piston 40 and the surrounding wall 36 of the hollow cylinder 34, which seal the fluid chamber 42.

A piston rod 54 connected to the piston 40 is provided on the side of the piston 40 facing away from the front wall 38. At an end section of a circumferential surface of the piston rod 54 facing away from the piston 40, an external thread 56 is formed on the piston rod 54. On both sides of the external thread 56, two gear wheels 58 and 60 that are part of the motor 32 are arranged, which engage with the external thread 56.

The gear wheels 58 and 60 are formed or adjusted with the external thread 56 in such a way that counter-rotation of the gear wheels 58 and 60 causes a translation of the piston rod 54 resp. the piston 40. Axes of rotation 62 and 64 extend perpendicular to an axial direction or a longitudinal axis of the piston rod 54. Rotation of the gear wheels 58 and 60 is caused by activation of the motor 32.

A coupling part 66 is provided on a front face of the piston rod 54 facing away from the piston 40, by means of which the piston rod 54 can be moved relative to the hollow cylinder 34. If the coupling part 66 is rotated by motor (in particular by way of the motor 32 connected to the coupling part 66) when gear wheels 58 and 60 are at a standstill, the engagement of gear wheels 58 and 60 in the external thread 56 causes the piston rod 54 to be moved relative to the axes of rotation 62 and 64 of gear wheels 58 and 60. The gear wheels 58 and 60 as well as the coupling part 66 are all driven by the motor 32. Alternatively, the device 12 may also be designed such that the gear wheel 58, the gear wheel 60 and/or the coupling part 66 are driven by a respectively separate motor.

In order to determine the position of the piston 40 relative to the hollow cylinder 34, the device 4 has a position sensor 68 connected to the control device 18 by way of signals (see FIG. 1). Based on a change in the position of the piston 40, the control device 18 can determine a quantity of fluid ejected or sucked in.

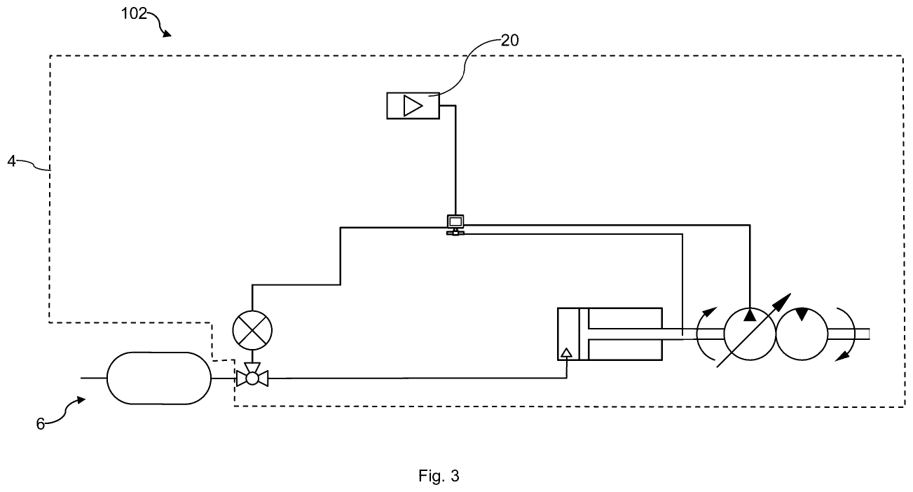

FIG. 3 shows a schematic view of a system 102 according to a second embodiment of the present disclosure. The system 102 according to the second embodiment differs from the system 2 according to the first embodiment only in that the system 102 according to the second embodiment has only a wired operating unit 20 and no radio-connected operating unit 22.

FIG. 4 shows a schematic view of a system 202 according to a third embodiment of the present disclosure. The system 202 according to the third embodiment differs from the system 2 according to the first embodiment only in that the system 202 according to the third embodiment has only a radio-connected operating unit 22 and no wired operating unit 20.

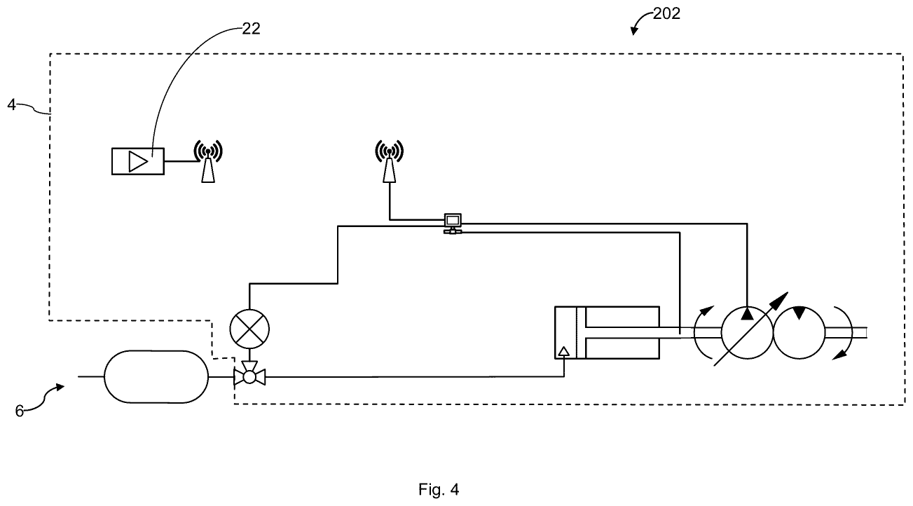

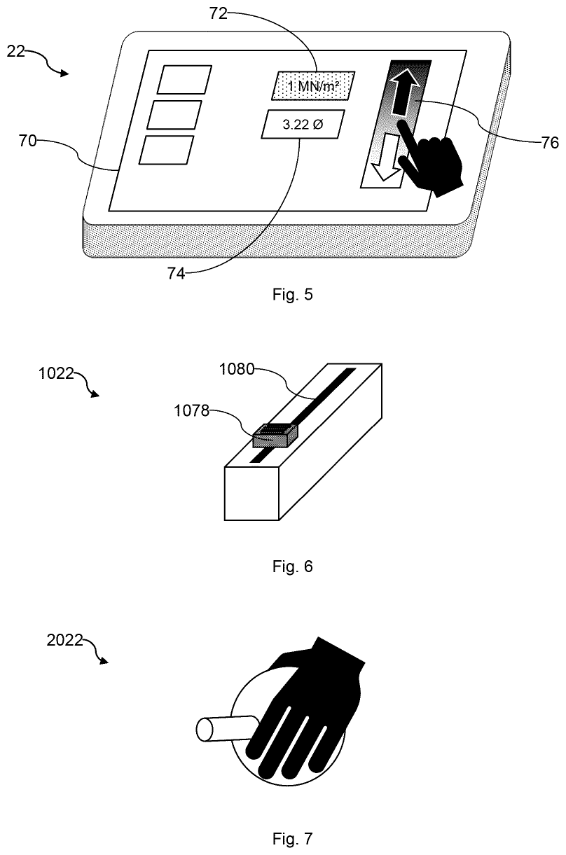

FIG. 5 shows a schematic view of an operating unit 22 according to a first embodiment. The operating unit 22 is designed in the form of a tablet that has a touch-sensitive screen 70. The operating unit 22 is adapted to display a field 72 on the touch-sensitive screen 70 in which a pressure value is indicated, a field 74 in which a diameter value of the balloon catheter 6 is indicated, and display a field 76 in which a slide switch is displayed. The operating unit 22 is configured such that a value in one of fields 72 or 74 can be changed by first activating one of fields 72 or 74 by touching it (see field 72 in FIG. 5) and subsequently changing the value of the activated field by swiping along field 76.

FIG. 6 shows a schematic view of an operating unit 1022 according to a second embodiment. The operating unit 1022 is designed as a slide switch and has a slide 1078 that can be moved along a linear guide 1080. The operating unit 1022 is adapted to specify a threshold value or target value for the pressure measured by the pressure sensor device 16 by moving the slide 1078 along the linear guide 1080 of the control device 18.

FIG. 7 shows a schematic view of an operating unit 2022 according to a third embodiment. The operating unit 2022 is designed in the form of a squeezable ball. The operating unit 2022 is adapted to specify a threshold value or target value for the pressure measured by the pressure sensor device 16, corresponding to the degree of compression of control device 18.

The operating units 22, 1022, and 2022 shown in FIGS. 5, 6, and 7 are designed as wireless operating units. Alternatively or in addition, it is also possible for the operating units 22, 1022, and 2022 to be configured so as to be connected to the control device 18 by way of a wire.

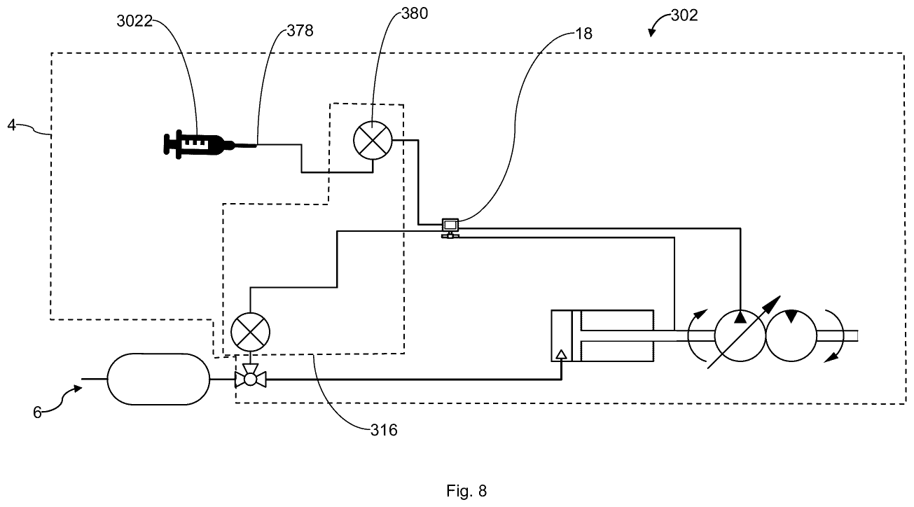

FIG. 8 shows a schematic view of a system 302 according to a fourth embodiment of the present disclosure. The system 302 according to the third embodiment differs from the systems 2, 102, and 202 according to the first three embodiments only in that the system 302 according to the fourth embodiment has a fluid-mechanical (i.e. a hydraulic or pneumatic) operating unit 3022 instead of an electric or electronic operating unit 20, 22, 1022 or 2022 and, corresponding to the fluid-mechanical operating unit 3022, is equipped with a pressure connection 378 and a pressure sensor 380, which is fluid-mechanically connected to the pressure connection 378 and connected by signals to the control device 18. Preferably, the pressure sensor 380 is designed as part of a pressure sensor unit 316 according to the disclosure.

The pressure connection 378 is adapted to be connected to the operating unit 3022.

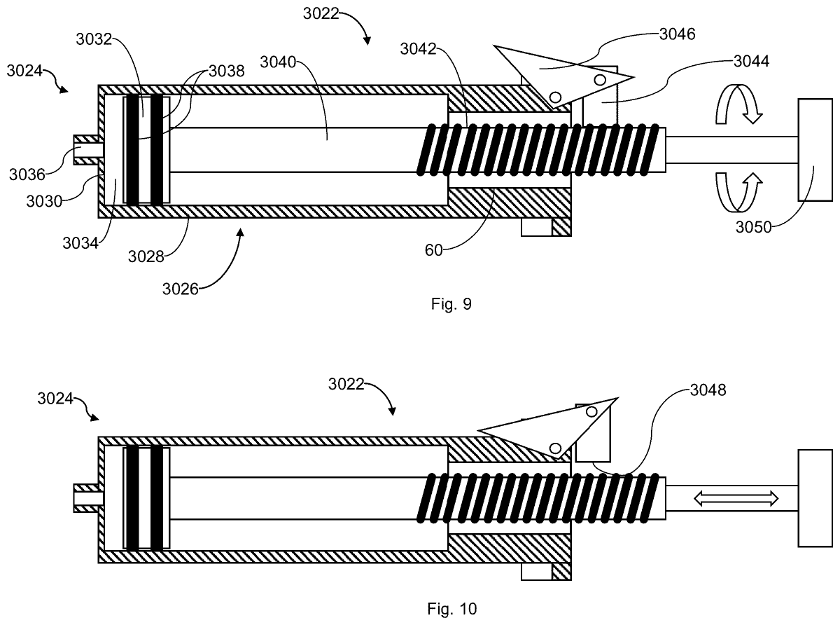

FIG. 9 shows a schematic view of the fluid-mechanical operating unit 3022 according to a first embodiment.

The operating unit 3022 is designed in the form of a manual inflation syringe 3024.

The inflation syringe 3024 has a hollow cylinder 3026 with a surrounding wall 3028. The hollow cylinder 3026 is open on one side and has a front wall 3030 on the side opposite the open side. At the open side of the hollow cylinder 3026, a piston 3032 is introduced into the hollow cylinder 3026. The piston 3032 is displaceable in the hollow cylinder 3026 and, together with surrounding wall 3028 and front wall 3030, encloses a fluid chamber 3034. The size of the fluid chamber 3034 is variable due to the displacement of the piston 3032 relative to the hollow cylinder 3026.

A fluid outlet 3036 is formed on the front wall 3030, which represents a control fluid interface according to the disclosure. The fluid outlet 3036 is fluid-mechanically connected or connectable to the pressure connection 378.

When the piston 3032 is moved toward the front wall 3030, fluid located in the fluid chamber 3034 (i.e., a control fluid as disclosed) is conveyed from the fluid outlet 3036 to the pressure connection 378. Moving the piston 40 toward the front wall 38 thus increases the pressure at the pressure connection 378.

If the piston 3032 is moved away from the front wall 3030, a negative pressure is generated at the fluid outlet 3036, which causes fluid to be sucked in from the pressure connection 378 and thus reduces the pressure at the pressure connection 378.

Seals 3038 are provided on a circumferential surface of the piston 3032 between the piston 3032 and the wall 3028 of the hollow cylinder 3026, which seal the fluid chamber 3034.

On the side of the piston 3032 facing away from the front wall 3030, a piston rod 3040 connected to the piston 3032 is provided. An external thread 3042 is formed on the piston rod 3040 at an end section of a circumferential surface of the piston rod 3040 facing away from the piston 3032. On the side of the hollow cylinder 3026 facing away from front wall 3030 a threaded block 3044 is connected to the hollow cylinder 3026 via a tilting lever 3046. The threaded block 3044 is connected to the tilting lever 3046 in an articulated or pivotable manner, and the tilting lever 3046 is connected to the hollow cylinder 3026 in an articulated or pivotable manner. The threaded block 3044 has a threaded section 3048 on a side facing the external thread 3042 of the piston rod (see FIG. 10), which is designed to be brought into engagement with the external thread 3042. The threaded block 3044 and the tilting lever 3046 together form a quick release valve, which is designed to be moved into a deactivated position by means of tilting lever 3046, in which the threaded block 3044 or its threaded section 3048 is engaged with external thread 3042 (see FIG. 9), and by way of tilting lever 3046 into an activated position, in which the threaded block 3044 or its threaded section 3048 is not engaged with the external thread 3042 (see FIG. 10). Preferably, the quick release mechanism resp. its tilting lever 3046 is preloaded by means of a spring (not shown) in such a way that the quick release mechanism is preloaded into its deactivated position and the quick release mechanism can only be moved into its activated position by overcoming the preload.

A rotary handle 3050 is provided on a front face of the piston rod 3040 facing away from the piston 3032, by means of which the piston rod 3040 can be moved manually relative to the hollow cylinder 3026. If the rotary handle 3050 is turned manually when the quick release valve is deactivated (see arrows in FIG. 9), the engagement of the threaded section 3048 in the external thread 3042 causes the piston rod 3040 to move relative to the threaded block 3044 and thus relative to the hollow cylinder 3026. When the quick release is in an activated position, the threaded block 3044 and the external thread 3042 are disengaged, so that the piston rod 3040 can be moved axially and the piston 3032 can be moved, in particular quicker, by moving the rotary handle 3050 in the axial direction of the piston 3032 (see double arrow in FIG. 10).

In order to operate device 4 in system 302 by means of operating unit 3022 the size of fluid chamber 3034 is varied by changing the axial position of piston 3032 relative to hollow cylinder 3026 by means of rotary handle 3050 (either with the quick release valve activated or deactivated), thereby varying the pressure of the fluid at the fluid outlet 3036 and thus at pressure connection 378. This pressure variation is measured by pressure sensor 380. The control device 18 of the device 4 of the system 302 is designed to convert the pressure measured at the pressure connection 378 resp. the pressure variation measured at the pressure connection 378 into a control command for the motor 32 of the fluid delivery device 12, so that a corresponding pressure resp. pressure variation is generated in the balloon catheter 6. “Converting” means in particular that the pressure generated by the fluid delivery device 12 is higher than the pressure generated manually by operating unit 3022 and/or that a pressure curve generated by the fluid delivery device 12 is smoothed compared to a pressure curve generated manually by operating unit 3022.

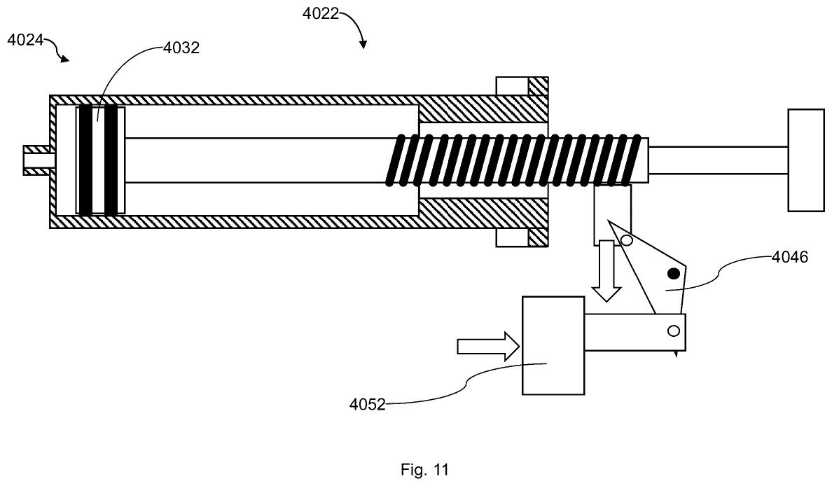

FIG. 11 shows a schematic view of a fluid-mechanical operating unit 4022 according to a second embodiment of the disclosure. The fluid-mechanical operating unit 4022 according to the second embodiment differs from fluid-mechanical operating unit 3022 according to the first embodiment in that a tilting lever 4046 of the fluid-mechanical operating unit 4022 according to the second embodiment is connected in an articulated or pivotable manner to a trigger 4052. By providing a trigger 4052, it is possible to provide a pistol grip (not shown) on an inflation syringe 4024 of operating unit 4022, thereby improving operability of operating unit 4022. Apart from the differences described, the fluid-mechanical operating unit 4022 according to the second embodiment corresponds to the fluid-mechanical operating unit 3022 according to the first embodiment.

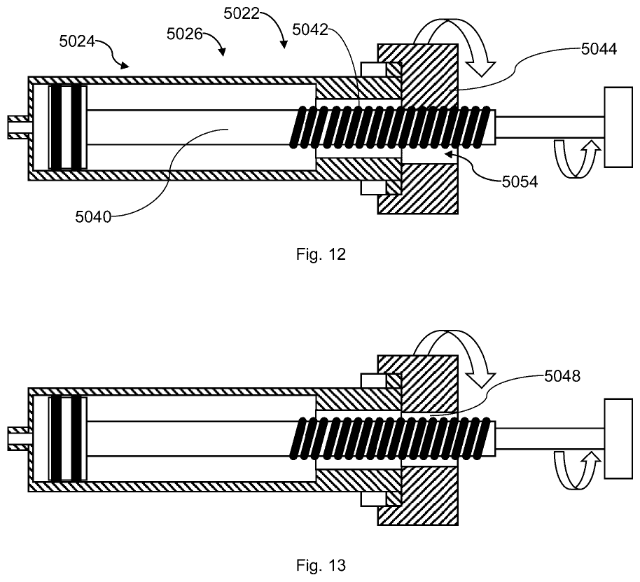

FIG. 12 shows a schematic view of a fluid-mechanical operating unit 5022 according to a third embodiment with a quick release valve in a deactivated position. FIG. 13 shows a schematic view of the fluid-mechanical operating unit 5022 according to the third embodiment with the quick release valve in an activated position.

The fluid-mechanical operating unit 5022 according to the third embodiment is distinguished from the fluid-mechanical operating unit 3022 according to the first embodiment in that that a threaded block 5044 is formed as an eccentric, which is rotatably mounted to a hollow cylinder 5024 such that an axis of rotation of the threaded block 5044 is parallel to a longitudinal axis of piston rod 5040. The threaded block 5044 has a recess 5054 through which the piston rod 5040 extends.

The recess 5054 is formed eccentrically such that an inner surface of the recess 5054, formed as a threaded section 5048, in a first position of threaded block 5044 relative to hollow cylinder 5024 (see FIG. 12), engages with an external thread 5042 of piston rod 5040 (see FIG. 12) and, in a twisted second position compared to the first position of threaded block 5044 relative to the hollow cylinder 5024, is not in engagement with external thread 5042 of the piston rod 5040 (see FIG. 13).

In the fluid-mechanical operating unit 5022 according to the third embodiment, the quick release mechanism is thus formed solely by threaded block 5044. A tilting lever as in the fluid-mechanical operating units 3022 and 4022 according to the first two embodiments is not necessary.

Apart from the afore-described differences, the fluid-mechanical operating unit 5022 according to the third embodiment corresponds to the fluid-mechanical operating unit 3022 according to the first embodiment.

### LIST OF REFERENCE SIGNS

2 System according to the first embodiment
4 Device
6 Balloon catheter
8 Catheter interface
10 Three-way valve
12 Fluid delivery device
14 Piston syringe
16 Pressure sensor device
18 Control device
20 Wired operating unit
22 Radio-connected operating unit
24 Catheter-side connection point of the three-way valve
26 Fluid delivery device-side connection point of the three-way valve
28 Pressure sensor device-side connection point of the three-way valve
30 Balloon
32 Motor of the fluid delivery device
34 Hollow cylinder of piston syringe
36 Surrounding wall of piston syringe
38 Front wall of piston syringe
40 Piston of piston syringe
42 Fluid chamber of piston syringe
44 Fluid outlet of piston syringe
52 Seal between piston and wall
54 Piston rod
56 External thread of piston rod
58, 60 Gear wheel of motor
62, 64 Axis of rotation
66 Coupling part on piston rod
68 Position sensor
70 Touch-sensitive screen
72 to 76 Field on touch-sensitive screen
102 System according to the second embodiment
202 System according to the third embodiment
302 System according to the fourth embodiment
378 Pressure connection
380 Pressure sensor
1022 Operating unit according to the second embodiment
1078 Slide
1080 Linear guide
2022 Electric operating unit according to third embodiment
3022 Fluid-mechanical operating unit according to first embodiment
3024 Inflation syringe
3026 Hollow cylinder of inflation syringe
3028 Wall of inflation syringe
3030 Front wall of inflation syringe
3032 Piston of inflation syringe
3034 Fluid chamber of inflation syringe
3036 Fluid outlet of inflation syringe
3038 Seal between piston and wall
3040 Piston rod
3042 External thread of piston rod
3044 Threaded block
3046 Tilting lever
3048 Threaded section
3050 Rotary handle on piston rod
4022 Fluid-mechanical operating unit according to the second embodiment
4024 Inflation syringe
4032 Piston of inflation syringe
4046 Tilting lever
4052 Trigger
5022 Fluid-mechanical operating unit according to the third embodiment
5024 Hollow cylinder of inflation syringe
5032 Piston of inflation syringe
5040 Piston rod
5042 External thread
5044 Threaded block
5048 Threaded section
5054 Recess

1.-15. (canceled)

16. A device for automatically inflating a balloon catheter with a fluid, the device comprising:
a catheter interface;
a fluid delivery device;
a pressure sensor device; and
a control device,
the catheter interface being connectable to the balloon catheter in a demountable manner so that the fluid is flowable from the device via the catheter interface into the balloon catheter,
the fluid delivery device being configured to eject the fluid at the catheter interface from the device by a motor,
the pressure sensor device being configured to measure a pressure of fluid ejected from the device,
the control device configured to control a quantity of the fluid ejected from the device by the fluid delivery device, based on the pressure of fluid measured by the pressure sensor device, by driving the motor of the fluid delivery device,
the fluid delivery device being configured to suck in the fluid into the device at the catheter interface by the motor, and by an energy storage device configured to store electrical or mechanical energy so that, during a power failure, excess pressure is reducible and a vacuum is creatable.

17. The device according to claim 16, wherein the control device is configured to cause the fluid to be sucked in via the fluid delivery device when a predetermined threshold value of the pressure of fluid measured by the pressure sensor device is exceeded.

18. The device according to claim 17, wherein the control device is configured to prevent the fluid from being sucked in via the fluid delivery device after the predetermined threshold value has been exceeded and a subsequent drop in the pressure of fluid measured by the pressure sensor device when the predetermined threshold value is reached again.

19. The device according to claim 16, wherein the fluid is a liquid and the device has a venting device by which a gas mixed with the liquid is dischargeable from the device.

20. The device according to claim 16, wherein the control device is configured to control the fluid delivery device in such a way that the fluid is delivered at different predetermined delivery rates.

21. The device according to claim 16, wherein the control device is configured to regulate the fluid delivery device depending on the pressure of fluid measured by the pressure sensor device.

22. The device according to claim 21, wherein the control device is configured to regulate the fluid delivery device in such a way that at least two different predetermined pressure levels are reached.

23. The device according to claim 16, wherein the motor of the fluid delivery device has a tensioner configured to be tensioned when the fluid is ejected and to be relaxed when the fluid is sucked in.

24. The device according to claim 16, wherein the control device has a data interface configured to transfer data to and/or from a smartphone, a tablet or another device.

25. The device according to claim 24, further comprising an operating unit connected to the data interface, the data interface configured to receive control commands from a person and transmit the control commands to the control device.

26. The device according to claim 25, wherein the operating unit is a manual inflation syringe.

27. The device according to claim 25, wherein:
the operating unit is an inflation syringe by which a control fluid is conveyable to a control fluid interface of the inflation syringe by moving a piston,
the data interface is a pressure connection that is connectable to the control fluid interface of the inflation syringe,
the pressure sensor device is configured to measure a pressure of the control fluid at the pressure connection, and
the control device is configured to control the fluid delivery device depending on the pressure of the control fluid measured at the pressure connection.

28. The device according to claim 25, wherein the operating unit has a display configured to display condition data of the device.

29. A system for performing an angioplasty, the system comprising:
the device according to claim 16; and
a balloon catheter.

30. The system according to claim 29, wherein the device is configured to generate a negative pressure or vacuum in the balloon catheter.
